(19) United States
(12) Patent Application Publication
(10) Pub. No.: US 2009/0117612 A1
(43) Pub. Date: May 7, 2009

(54) METHOD OF BIOOXIDATION USING AN OLD YELLOW ENZYME

(76) Inventors: Anton Glieder, Gleisdorf (AT); Mattias Schittmayer, Graz (AT)

Correspondence Address: CHRISTIE, PARKER & HALE, LLP, PO BOX 7068, PASADENA, CA, 91109-7068, US

(21) Appl. No.: 12/114,665

(22) Filed: May 2, 2008

Related U.S. Application Data
(60) Provisional application No. 60/915,581, filed on May 2, 2007.

(51) Int. Cl. C12P 33/06; C12N 9/14

(52) U.S. Cl. 435/58; 435/195

(57) ABSTRACT

A method for a chemoselective and regioselective enzyme mediated oxidation of carbon-hydrogen bonds of substrates using a Geobacillus kaustophilus ‘Old Yellow Enzyme’ is provided. It is shown that OYEs can be used to facilitate the bioxidation of substrates, such as testosterone. It is also shown that the use of OYEs allows for the production of oxidized substrates in one step reactions, which are otherwise not accessible or only accessible after complex and inefficient multi-step reactions. In addition, the OYE used shows high stability at high temperature. An exemplary embodiment is provided showing the use of an OYE to convert testosterone to 6α-hydroxytestosterone.

### CROSS-REFERENCE TO RELATED APPLICATION

This application claims the benefit of U.S. Provisional Application No. 60/915,581, filed May 2, 2007 the disclosure of which is incorporated herein by reference.

### FIELD OF THE INVENTION

The current invention is directed to an enzymatic catalysis utilizing an old yellow enzyme; and more particularly to a method of hydroxylating testosterone using an old yellow enzyme.

### BACKGROUND OF THE INVENTION

The biooxidation of substrates is of high interest because of the importance of the various metabolites that can be formed from such reactions. For example, oxidative metabolism of testosterone by human liver microsomes allows for the formation of 1β-, 2α-/β-, 6β-, 15β-, and 16β-hydroxytestosterones, which are important metabolites for the body.

Usually monooxygenases are used for this kind of reactions, but these compounds are not available for all substrates. Among the monooxygenases, cytochrome P450 (P450) enzymes, a superfamily of more than 160 known members, are also responsible for the biosynthesis or catabolism of steroid hormones, including the oxidative metabolism of endogenous and exogenous testosterone. (See, e.g., Wood A W, Swinney D C, Thomas P E, Ryan D E, Hall P F, Levin W, and Garland W A (1988) Mechanism of androstenedione formation from testosterone and epitestosterone catalyzed by purified cytochrome P-450b. J Biol Chem 263: 17322-17332; Yamazaki H and Shimada T (1997), Progesterone and testosterone hydroxylation by cytochromes P450, 2C19, 2C9, and 3A4 in human liver microsomes. Arch Biochem Biophys 346: 161-169; and Rendic S, Nolteemsting E, and Schänzer W (1999) Metabolism of anabolic steroids by recombinant human cytochrome P450 enzymes. J Chromatogr Biomed Appl 735: 73-83). In these reactions, 13-hydroxylation at either the C6 or C16 position is the major route of testosterone oxidative metabolism. Human liver enzymes are also found to oxidize testosterone at the C17 position to form androstenedione.

However, conventional testosterone oxidation enzymes, such as P450 enzymes, are usually very unstable. In addition, they are not available for all substrates. Accordingly, there remains a need to find improved and more efficient oxidative enzymes for the hydroxylation of testosterone.

The present invention addresses this need for an improved method for the biooxidation of testosterone, without the disadvantages of conventional biocatalytic enzymes such as monooxygenases.

### SUMMARY OF THE INVENTION

The current invention is directed to a method of the chemoselective and regioselective oxidation of carbon-hydrogen bonds using an enzymatic reaction. In one embodiment, the invention is directed to a method of hydroxylating testosterone using an isolated Old Yellow Enzyme. In another embodiment, the invention is directed to an isolated old yellow enzyme capable of hydroxylating testosterone.

### BRIEF DESCRIPTION OF THE DRAWINGS

These and other features, aspects, and advantages of the present invention will be more fully understood when considered with respect to the following detailed description, appended claims, and accompanying drawings, wherein:

FIG. 1 is a reaction diagram of the hydroxylation of testosterone to 6α and 6β-hydroxytestosterone;

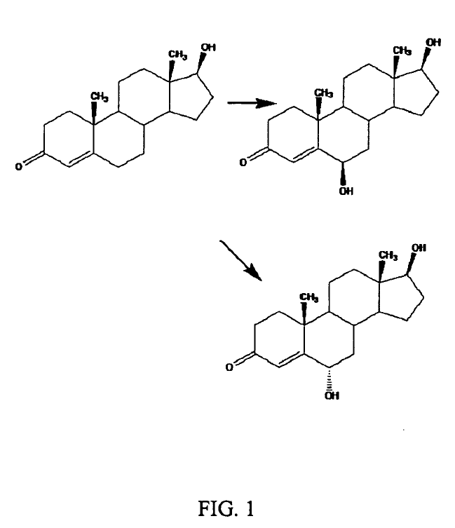

FIG. 2 shows the ¹H-NMR spectrum of 6α-hydroxytestosterone;

FIG. 3 shows the ¹H-NMR spectrum of 6β-hydroxytestosterone;

FIG. 4 shows the OYE expressed in different E. Coli expression strains as 38 kDalton bands;

FIG. 5 shows the OYE expressed in DH5α cells as a 38 kDalton band, which is absent in the negative control lane; and

FIG. 6 shows the testosterone conversion into 6α-hydroxytestosterone as measured by HPLC-MS.

### DETAILED DESCRIPTION OF THE INVENTION

The current invention is directed to a method for chemoselective and regioselective bioxidation of carbon-hydrogen bonds of substrates using a Geobacillus kaustophilus ‘Old Yellow Enzyme’, referred to hereinafter as “OYE”.

Applicants have discovered that, surprisingly, OYEs can be used to facilitate the biooxidation of substrates, such as testosterone. It has been further discovered that the use of OYE allows for the production of oxidized substrates in one-step reactions, which are otherwise not accessible or only accessible after complex and inefficient multi-step reactions. For instance, conventional enzymes such as human cytochromes are also capable to catalyze the oxidation reaction in one step but they are not stable and thus difficult to apply on a preparative scale. On the other hand, chemical syntheses which could be used for preparative scales usually involve multiple steps and are non-selective, thus requiring subsequent, sophisticated preparative-scale chromatographic product separation.

Thus, in a first aspect, the invention provides a method for enzyme-mediated oxidation of a substrate and comprises contacting the substrate with an Old Yellow Enzyme (OYE) to form an oxidation product thereof.

In another aspect, the invention provides a method for enzyme-mediated hydroxylation of testosterone into 6α and 6β hydroxytestosterone.

In another aspect, the invention provides an isolated Old Yellow Enzyme (OYE) capable of catalyzing an oxidation reaction of a substrate into oxidation products thereof. For example, one such OYE is capable of hydroxylating testosterone to 6α and 6β hydroxytestosterone.

In addition, the OYE used shows high stability. For example, the activity assays for hydroxylation using OYE are done at 55° C. for up to 48 hours, suggesting that the OYE is very stable at that high temperature.

In the course of screening for new microbial hydroxylating activities, two strains are identified, both oxidizing testosterone to 6α and 6β hydroxytestosterone. The two strains correspond to Geobacillus thermoglucosidasius and Geobacillus kaustophilus. There are also literature reports about P450 enzyme(s) from Geobacillus which can perform the hydroxylation of testosterone.

Protein purification from Geobacillus thermoglucosidasius and Geobacillus kaustophilus crude lysates and subsequent fingerprinting by Matrix-Assisted Laser Desorption/Ionization-Time-Of-Flight (MALDI-TOF) are chosen as a strategy for identifying the testosterone hydroxylating activity.

To identify the enzyme responsible for the hydroxylating activity, Geobacillus thermoglucosidasius DSM 2542 and Geobacillus kaustophilus DSM 7263, the latter one having the advantage of a sequenced genome, are obtained from Deutsche Sammlung von Mikroorganismen und Zellkulturen GmbH (DSMZ; German Collection of Microorganisms and Cell Cultures) and tested for their ability to hydroxylate testosterone.

The hydroxylation activity is preferably determined by measuring the oxidation of testosterone (5 mM), in the presence of NADPH (0.5 mM), into 6α-hydroxytestosterone at 55° C. High-Performance Liquid Chromatography/Mass Spectrometry (“HPLC/MS”) analysis is used to detect the production of 6α-hydroxytestosterone after 24 h, and a further increase in product yield after 48 h. After 48 h the reaction was stopped.

To purify the enzyme of interest, the raw lysates of the strain Geobacillus kaustophilus DSM 7263, whose genome sequence data is available, are subjected to anion exchange chromatography followed by size exclusion chromatography. Several active fractions are obtained and loaded onto a Sodium Dodecyl Sulfate Polyacrylamide Gel Electrophoresis (“SDS-PAGE”) and, after gel electrophoresis, stained with Coomassie blue. One lane yields a single band of approximately 40 kDa, which is also present in all other active fractions. This band is isolated from the gel and prepared for fingerprinting by Matrix-Assisted Laser Desorption/Ionization-Time-Of-Flight (MALDI-TOF). MALDI-TOF analyses and subsequent database search with the acquired fingerprint yield, amongst others, OYE of Geobacillus kaustophilus, YP 148185.1

Comparing the sequence of the active purified protein YP 148185.1 against the genome sequence of Geobacillus kaustophilus facilitates the identification of the gene coding for YP 148185.1.

Primers are designed to amplify OYE from Geobacillus kaustophilus genomic DNA by polymerase chain reaction (PCR). PCR products yield a band at the expected size of approximately 1020 bp. The fragment is cloned into an expression vector (pEamTA) yielding pEamTAOYE. The sequence of the expressed protein confirmed that the cloned fragment is indeed the desired OYE.

For further expression of OYE, the DNA fragment of interest is transformed into a DH5α strain of E. coli using transformation procedures that are well known in the art.

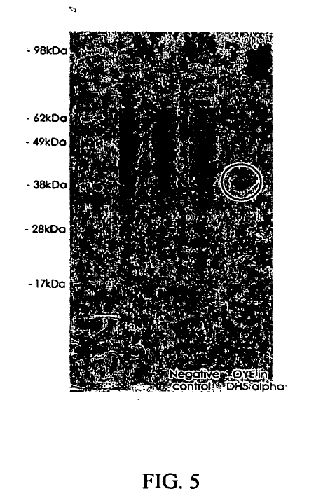

After expression in E. coli DH5α, the cells are harvested, ruptured, and centrifuged, and loaded onto SDS-PAGE. A thick band visible in the soluble fraction at the expected size of 38 kDa is observed. A small amount of OYE is also found to remain in the insoluble fraction. A negative control (pEamTA in DH5α) does not show a band at 38 kDa (FIG. 5).

Hydroxylation activity assay as described above is carried out to confirm that 38 kDa band is capable of converting testosterone to 6α-hydroxytestosterone.

These results confirm that OYEs can be used to facilitate the biooxidation of substrates, such as testosterone. It has been further discovered that the use of OYE allows for the production of oxidized substrates in one-step reactions at high yield. In addition, the OYE used shows high stability at high temperature.

The following are non-limiting examples of the invention.

### EXAMPLE 1

Hydroxylation of testosterone by cell lysates of Geobacillus thermoglucosidasius DSM 2542 and Geobacillus kaustophilus DSM 7263.

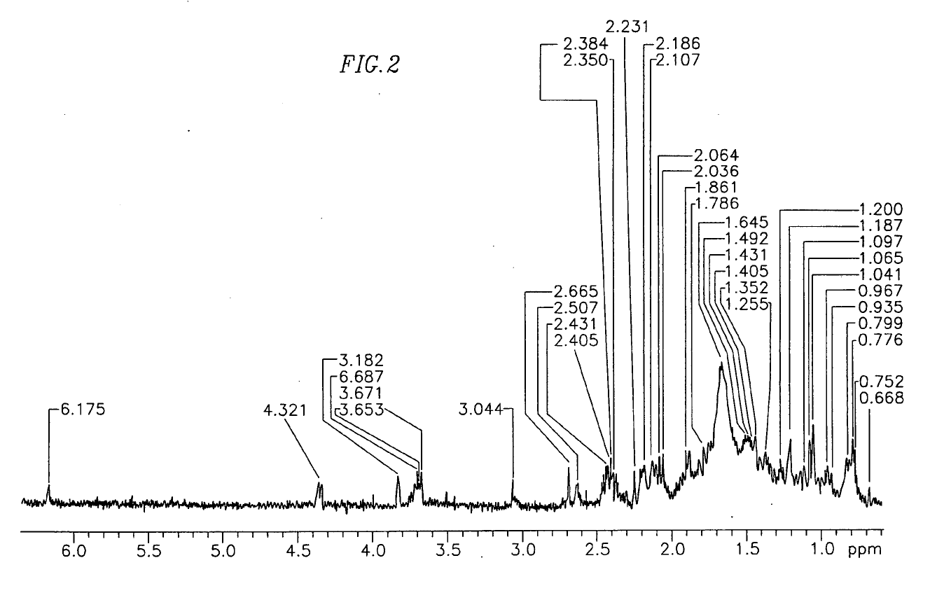

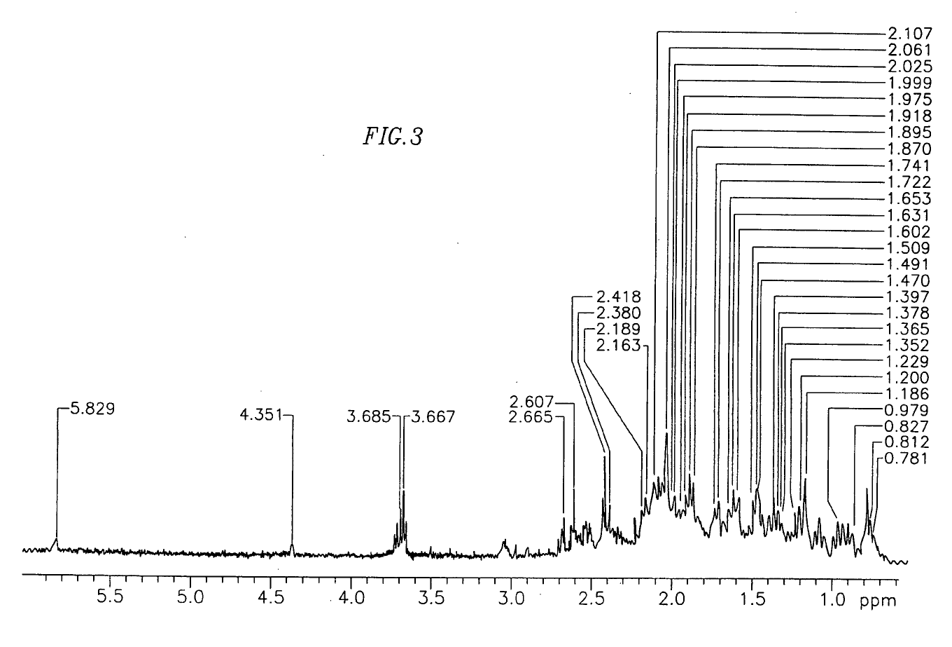

Geobacillus thermoglucosidasius DSM 2542 and Geobacillus kaustophilus DSM 7263 were obtained from Deutsche Sammlung von Mikroorganismen und Zellkulturen GmbH (DSMZ; German Collection of Microorganisms and Cell Cultures) and tested for their ability to hydroxylate testosterone. Raw lysates of both strains were analyzed by HPLC/MS and showed conversion to two products which both showed a m/z ratio of 305, as expected for hydroxylated testosterone, but with different retention times. While one of the metabolites corresponded exactly with an authentic 6β-hydroxytestosterone standard, the hydroxylation position of the second metabolite was unclear in the beginning. Both metabolites were then prepared on a milligram scale for ¹H-NMR analysis. The NMR spectra are shown in FIGS. 2 and 3. Peak 1 in FIG. 2 was identified as 6α-hydroxytestosterone and Peak 2 of FIG. 3 was identified as 6β-hydroxytestosterone.

### EXAMPLE 2

### Expression of OYE in Different Strains of E. Coli

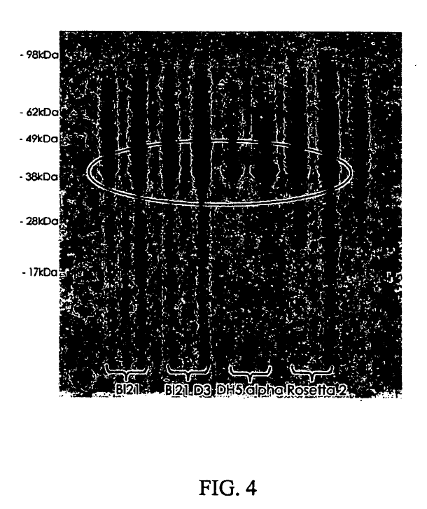

For further expression of OYE, new expression strains of E. Coli were chosen. 2 μL of OYE-DNA were transformed in the strains listed below.
Rosetta
Rosetta 2
B121
B121 D3
DH5α
   All cells except for DH5α were electrocompetent cells according to transformation procedures that are well known in the art. The regenerated cell suspension were plated out on LB-Amp-plates (100 μg/mL), except for the cells of the Rosetta strains, which were plated on LB-AMP-Chloramphenicol plates (100 μg/mL). After an incubation period of about 24 hours at 37° C., the grown colonies were used for inoculation of 100 mL LB-Amp and LB-AMP-Chloramphenicol, respectively. No growth on agar plates after transformation was recorded for the Rosetta cells and almost no colonies had been obtained by using the Rosetta 2 cells. The inoculated flasks were shaken constantly with 120 rpm at 28° C. After 5 hours at an optical density of 0.5, protein expression was induced by adding 0.5 mM IPTG to each flask. The temperature was then lowered to 20° C. The cells were harvested, ruptured, and ultracentrifuged and the supernatants were loaded on SDS-PAGE. FIG. 4 shows the expression of OYE in different expression E. coli strains. Using DH5α cells for expression of OYE gave the best results, followed by Rosetta 2.

### EXAMPLE 3

### Expression of OYE by DH5α Cells

As a result of the limited number of transformants by using the Rosetta 2 cells, for further expression procedures of OYE only the chemical competent DH5α cells were used. FIG. 5 shows the expression of OYE in DH5α cells only. DH5α cells transformed with the vector pEamTA (without the OYE fragment) were used as a negative control. A 38 kDalton band was obtained in the OYE transformed DH5α, but was absent in the negative control.

### EXAMPLE 4

### Hydroxylation Activity of the OYE Containing Fraction

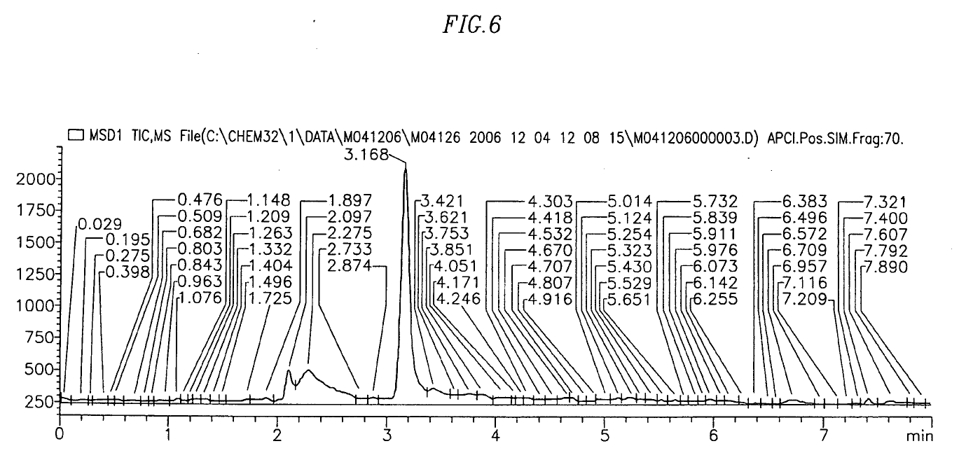

To verify that the fraction that yielded a 38 kDalton band on the gel as shown in FIG. 5 contains hydroxylating capability, the fraction was tested for hydroxylation activity wherein the testoterone conversion was analyzed by HPLC-MS. FIG. 6 shows a peak corresponding to the formation of 6α-hydroxytestosterone.

Those skilled in the art will appreciate that the foregoing examples and descriptions of various preferred embodiments of the present invention are merely illustrative of the invention as a whole, and that variations in the methodology of the present invention may be made and fall within the scope of the invention. Accordingly, the present invention is not limited to the specific embodiments described herein but, rather, is defined by the scope of the appended claims and equivalents thereof.

What is claimed is:

1. A method for enzyme-mediated oxidation of a substrate comprising:
contacting the substrate with an Old Yellow Enzyme (OYE) to form an oxidation product thereof.

2. The method of claim 1, wherein the oxidation is a hydroxylation reaction.

3. The method of claim 2, wherein the substrate is testosterone.

4. The method of claim 3, wherein the oxidation product is one of either a 6α-hydroxytestosterone or a 6β-hydroxytestosterone.

5. The method of claim 1, wherein the oxidation of the substrate is a one step reaction.

6. The method of claim 2, wherein the OYE shows stability at temperatures higher than 45° C.

7. A method for enzyme-mediated hydroxylation of testosterone comprising:
contacting testosterone with an Old Yellow Enzyme (OYE) to form one of either a 6α-hydroxytestosterone or a 6β-hydroxytestosterone.

8. An isolated Old Yellow Enzyme (OYE) capable of catalyzing an oxidation reaction of a substrate into oxidation products thereof.

9. The old yellow enzyme of claim 8, wherein the oxidation reaction is a hydroxylation reaction.

10. The Old Yellow Enzyme of claim 9, wherein the substrate is testosterone.

11. The Old Yellow Enzyme of claim 10, wherein the substrate testosterone is hydroxylated to one of either a 6α-hydroxytestosterone or a 6β-hydroxytestosterone.

12. The Old Yellow Enzyme of claim 8, wherein the oxidation reaction is a one-step reaction.

13. The Old Yellow Enzyme of claim 8, wherein the OYE shows stability at temperatures higher than 45° C.

14. An isolated Old Yellow Enzyme (OYE) capable of catalyzing a hydroxylation reaction of testosterone to one of either a 6α-hydroxytestosterone or a 6β-hydroxytestosterone.
